(19) United States
(12) Patent Application Publication
(10) Pub. No.: US 2004/0191203 A1
(43) Pub. Date: Sep. 30, 2004

(54) Cosmetic or dermatological composition comprising n-acylaminoamide derivatives

(76) Inventor: Yann Mahe, Morsang sur Orge (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/482,282

(22) PCT Filed: Jun. 26, 2002
(86) PCT No.: PCT/FR02/02221

(30) Foreign Application Priority Data
Jun. 26, 2001 (FR) 01 08435

(51) Int. Cl. A61K 7/06; A61K 7/11

(52) U.S. Cl. 424/70.14

(57) ABSTRACT

Cosmetic or dermatological composition characterized in that it comprises an association between an elastase inhibiting compound of the N-acylaminoamide family and at least one compound for reducing hair loss and/or enhancing hair regrowth.

The invention relates to cosmetic or dermatological compositions adapted for treating the hair scalp and the hair more particularly with a view to reducing the loss thereof.

The man of the art has known for a long time that the natural hair loss, in man, overall reflects the hair follicle balance between the alternative growth phases (anagen phase) and the loss phases (telogen phase). The average ratio of the number of follicles in anagen phase to that in telogen phase is of the order of 9 (90/10). The follicle percentage being in a rest phase (catagen phase) therein appears to be very low.

The natural hair fall or loss could be estimated, on average, to a few hundred hair a day for a normal physiological condition. For an alopecic condition, it could reach up to several hundreds a day, leading to a moderate to important thinning in the hair scalp.

On the other hand, there is at the hair scalp surface, a microbiological flora naturally made of bacteria and yeasts. When an imbalance occurs in the natural composition of such a flora, the hair loss can be increased.

It is further known that some factors such as a hormonal imbalance, a physiological stress, dietary deficiencies, can accelerate the phenomenon.

Upon the alopecia, micro-inflammatory areas appear in the hair follicle. During this process, numerous inflammation mediators are released by the various cell subtypes such as the keratinocytes of the external and internal sheaths and/or the matrix, or even in some cases by the papilla fibroblasts.

Amongst those mediators chemo-attracting factors are released, being some kind of “biological recruiting agents” having the ability to attract some inflammatory cells of the blood compartment towards the cutaneous tissue. Such cells are then responsible for maintaining a local irritation.

It is known that during a superficial cutaneous stress, which can be more particularly chemical, physical or bacterial, the follicle keratinocytes release biological mediators having the ability to attract some skin infiltrative cells, in their turn, responsible for maintaining a transitory local irritation.

The biological mediators likely to be produced by the so-stressed keratinocytes may include chemokines which are chemo-attracting cytokines responsible for recruiting leukocytes on the inflammatory sites, including the interleukin 8 (IL-8) which is more particularly responsible for recruiting neutrophils Such cells infiltrating the irritated or aggressed areas then release enzymes including the leukocyte elastase.

Under the action of such an enzyme in particular, the extracellular supporting elastic fibres of the connective tissue can be damaged and consequently lead to a decrease of the skin elasticity and of the peribulbar environment.

It is also additionally known that synergistically with cathepsin G, the leukocyte elastase can dissociate the epiderm integrity widening the interkeratinocyte intercellular spaces.

Specially for the hair scalp, when the damage is very important, the superficial thin elastic fibres perpendicular to the skin surface disappear and the deeper thick fibres parallel to the skin surface break up. This is the so-called actinic elastosis.

During a cutaneous stress (chemical, physical, bacterial or neurogeneous), the keratinocytes release biological mediators (called chemo-attracting factors) having the ability to attract some infiltrative cells from the blood compartment towards the cutaneous tissue. Such cells are responsible for generating and later for maintaining a local irritation.

Amongst the chemo-attracting factors likely to be produced by the stressed keratinocytes, the interleukin 8 (IL-8) is more specifically responsible for recruiting polynuclear neutrophils. Such cells infiltrating the irritated or aggressed areas then release enzymes including the leukocyte elastase and other proteases (metalloproteinases, serine proteases, etc.).

Under the action of such an enzyme, the extracellular supporting elastic fibres of the connective tissue are damaged. Synergistically with cathepsin G, the leukocyte elastase can even dissociate the epiderm integrity widening the interkeratinocyte intercellular spaces (Ludolph-Hauser et al. Exp. Dermatol. 1999 8(1) 46-52). The leukocyte elastase has been recently accused of maintaining bedsores and for the occurrence of legs' venous ulcers, through its fibronectin altering activity (Herrick S. et al., Lab.Invest., 1997(3) 281-288. The sum of the altering localized micro-stresses (resulting, for example, from an extended exposure to the sun) can lead, in long term, to an accelerated loss of the skin natural elasticity. The network of elastic fibres of the underlying connective tissue and of the extracellular spaces is then progressively dismantled. Such an accelerated alteration could be cumulated to the normal ageing process of the skin characterized by a higher sensitivity of the elastic fibres to the elastase action (Stadler R & Orfanos CE Arch. Dermatol. Res. 1978 262 (1) 97-111.

It is known in the state of the art to provide, into the cutaneous tissue, molecules able to slow down the altering activity of the elastic fibres of the intercellular spaces.

In particular, it is known in the state of the art to provide at the alopecic area level, an agent limiting the hair loss. Those cosmetic and/or dermatological ingredients can include vitamins, biotin, Aminexil, minoxidil, sweet energetic agents, ginseng, KPV tripeptide and the salts thereof, etc. There are also solutions involved systemically on the androgen metabolism (Finasteride®).

The technical solution according to the invention comprises associating to the anti-loss/regrowth active ingredients, whatever their administration mode and their action mode, an inhibitor of the elastase activity able to slow down as well the altering activity of the elastic fibres of the hair scalp intercellular spaces.

Consequently, an object of the invention is a cosmetic or dennatological composition characterized in that it comprises an association between an elastase inhibiting compound of the N-acylaminoamide family and at least one compound reducing the hair loss and/or enhancing the hair regrowth.

Another object of the invention comprises a method for cosmetically treating the hair and/or hair scalp, characterized by applying on the hair and/or on hair scalp a composition such as defined hereinabove.

It has indeed been found that the compounds of formula (I) showed an activity inhibiting the elastase activity, and that they consequently were able to be used for limiting and/or overcoming the elastic fibre alteration.

As a result, they may be used in or for preparing a composition, the compounds or the composition being adapted for treating, either in a preventive and/or curative way, the elastic fibre alteration of the intercellular spaces, more particularly the hair scalp.

The novel association of N-acylaminoamide compounds with at least one compound reducing the hair loss and/or enhancing the hair regrowth makes it possible to significantly reinforce the anti-ageing effect of the matrix tissue, through providing a hair anti-loss effect or a hair regrowth effect.

According to the invention, the element regulating the elastase activity, the N-acyloaminoamide compound, and preferably a compound of the formula (1), is combined with one or more active ingredients able to reduce the hair loss or to enhance the hair regrowth.

The resulting composition is adapted for treating alopecic disorders and/or hair regrowth disorders.

Preferably, such a novel association is used in care and/or hygiene cosmetic preparations for the areas exposed to the sun (scalp), in care and/or hygiene cosmetic preparations for the hair areas and, generally, in all the so-called “anti-ageing” cosmetic preparations with the aim of slowing down the hair loss or enhancing their regrowth.

Without wishing to be bound to any theory, the Applicants contemplate that providing, at the level of the keratinocytes of the superficial layers of the skin and/or the peribulbar region, compounds able to slow down the altering activity of the elastic fibres can make it possible to reduce such an accelerated ageing phenomenon of the skin, resulting from superficial cutaneous stresses and that associating such compounds with at least one compound reducing the hair loss and/or enhancing the hair regrowth considerably reinforces their effects.

Preferred N-acylaminoamide Compounds

The compounds able to be used in this invention are thus represented by the following formula (I):

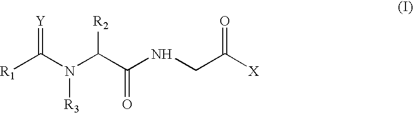

wherein

the Y moiety represents O or S,

the R1 moiety represents:

(i) a hydrogen atom;

(ii) an unsaturated or saturated, cyclic, branched or linear, hydrocarbon moiety, having 1 to 18 carbon atoms, optionally substituted by 1 to 5 identical or different groups selected amongst —OH; —OR; —O—COR; —SH; —SR; —S—COR; —NH₂; —NHR; —NRR′; —NH—COR; —Hal (halogen); —CN; —COOR; —COR; —P(O)—(OR)₂; —SO₂—OR;

with R and R′ representing, independently from one another, a linear, branched or cyclic, saturated or unsaturated hydrocarbon moiety having 1 to 6 carbon atoms, optionally halogenated, or even perhalogenated, said R and R′ moieties being able to form together with N, a 5 to 6 member carbon ring likely to additionally comprise one heteroatom selected amongst O, N and/or S in the ring, and/or likely to be substituted by 1 to 5 groups, whether identical or different, selected amongst —OH; —OR″; —O—COR″; —SH; —SR″; —S—COR″; —NH₂; —NHR″; —NH—COR″; —Hal (halogen); —CN; —COOR″; —COR″; with R″ representing, independently from one another, a linear, branched or cyclic, saturated or unsaturated hydrocarbon moiety having 1 to 6 carbon atoms, optionally halogenated, or even perhalogenated;

(iii) a moiety selected amongst the following moieties : —OR; —NH₂; —NHR; —NRR′; —NH—COR; —COOR; —COR;

with R and R′ representing, independently from one another, a linear, branched or cyclic, saturated or unsaturated hydrocarbon moiety having 1 to 6 carbon atoms, optionally halogenated, or even perhalogenated;

said R and R′ moieties being able to form together with N, a 5 to 6 member carbon ring likely to additionally comprise at least one heteroatom selected amongst O, N and/or S in the ring, and/or likely to be substituted by 1 to 5 identical or different groups selected amongst —OH; —OR″; —O—COR″; —SH; —SR″; —S—COR″; —NH₂; —NHR″; —NH—COR″; —Hal (halogen); —CN; —COOR″; —COR″; with R″ representing a linear, branched or cyclic, saturated or unsaturated hydrocarbon ring having 1 to 6 carbon atoms, optionally halogenated, or even perhalogenated;

the R2 moiety represents a linear, branched or cyclic, saturated or unsaturated hydrocarbon moiety having 1 to 18 carbon atoms;

optionally substituted by 1 to 5 identical or different groups selected amongst —OH; —OR; —O—COR; —SH; —SR; —S—COR; —NH₂; —NHR; —NRR′; —NH—COR; —Hal (halogen); —CN; —COOR; —COR;

with R and R′ representing, independently from each other, a linear, branched or cyclic, saturated or unsaturated hydrocarbon moiety having 1 to 6 carbon atoms, optionally halogenated, or even perhalogenated;

said R et R′ moieties being able to form together with N, a 5 to 6 member carbon ring likely to additionally comprise at least one heteroatom selected amongst O, N and/or S in the ring, and/or likely to be substituted by 1 to 5 identical or different groups selected amongst —OH; —OR″; —O—COR″; —SH; —SR″; —S—COR″; —NH₂; —NHR″; —NH—COR″; —Hal (halogen); —CN; —COOR″; —COR″; with R″ representing a linear, branched or cyclic, saturated or unsaturated hydrocarbon moiety having 1 to 6 carbon atoms, optionally halogenated, or even perhalogenated;

the R3 moiety represents a moiety selected amongst of the formula (II) or (III):

—A—C₆H_{(5-y)}—B_{y} (II)

—C₆H_{(5-y′)}—B_{y′} (III)

wherein

y is an integer between 0 and 5 inclusive, and y′ is an integer between 1 and 5 inclusive;

A is a linear or branched, saturated or unsaturated divalent hydrocarbon moiety having 1 to 18 carbon atoms;

optionally substituted by 1 to 5 identical or different groups selected amongst —OH; —OR; —O—COR; —SH; —SR; —S—COR; —NH₂; —NHR; —NRR′; —NH—COR; —Hal (halogen or even perhalogen); —CN; —COOR; —COR; —NO₂; —SO₂—OR;

with R and R′ representing, independently from each other, a linear, branched or cyclic, saturated or unsaturated hydrocarbon moiety having 1 to 6 carbon atoms, optionally halogenated, or even perhalogenated; said R et R′ moieties being able to form together with N, a 5 to 6 member carbon ring likely to additionally comprise at least one heteroatom selected amongst O, N and/or S in the ring, and/or likely to be substituted by 1 to 5 identical or different groups selected amongst —OH; —OR″; —O—COR″; —SH; —SR″; —S—COR″; —NH₂; —NHR″; —NH—COR″; —Hal (halogen); —CN; —COOR″; —COR″; with R″ representing a linear, branched or cyclic, saturated or unsaturated hydrocarbon moiety having 1 to 6 carbon atoms, optionally halogenated, or even perhalogenated;

B is a linear or branched, saturated or unsaturated hydrocarbon moiety having 1 to 18 carbon atoms;

optionally substituted by 1 to 5 identical or different groups selected amongst —OH; —OR; —O—COR; —SH; —SR; —S—COR; —NH₂; —NHR; —NRR′; —NH—COR; —Hal (halogen or perhalogen); —CN; —COOR; —COR; —NO₂; —SO₂—OR; with R and R′ representing, independently from each other, a linear, branched or cyclic, saturated or unsaturated hydrocarbon moiety having 1 to 6 carbon atoms, optionally halogenated, or even perhalogenated;

said R et R′ moieties being able to form together with N, a 5 to 6 member carbon ring likely to additionally comprise at least one heteroatom selected amongst O, N and/or S in the ring, and/or likely to be substituted by 1 to 5 identical or different groups selected amongst —OH; —OR″; —O—COR″; —SH; —SR″; —S—COR″; —NH₂; —NHR″; —NH—COR″; —Hal (halogen); —CN; —COOR″; —COR″; with R″ representing a linear, branched or cyclic, saturated or unsaturated hydrocarbon moiety having 1 to 6 carbon atoms, optionally halogenated, or even perhalogenated;

the X moiety represents a moiety selected amongst —OH; —OR₄; —NH₂; —NHR₄; —NR₄R₅, —SR₄; —COOR₄; —COR₄;

with R₄ and R₅ representing, independently from one another, a linear, cyclic or branched, saturated or unsaturated hydrocarbon moiety having 1 to 6 carbon atoms, optionally substituted by 1 to 5 identical or different groups selected amongst —OH; —OR; —O—COR; —SH; —SR; —S—COR; —NH₂; —NHR; —NRR′; —NH—COR; —Hal (halogen or even perhalogen); —CN; —COOR; —COR; with R and R′ representing, independently from each other, a linear, branched or cyclic, saturated or unsaturated hydrocarbon moiety having 1 to 6 carbon atoms, optionally halogenated, or even perhalogenated; said R et R′ moieties being able to form together with N, a 5 to 6 member carbon ring likely to additionally comprise at least one heteroatom selected amongst O, N and/or S in the ring, and/or likely to be substituted by 1 to 5 identical or different groups selected amongst —OH; —OR″; —O—COR″; —SH; —SR″; —S—COR″; —NH₂; —NHR″; —NH—COR″; —Hal (halogen); —CN; —COOR″; —COR″, with R″ representing a linear, branched or cyclic, saturated or unsaturated hydrocarbon moiety having 1 to 6 carbon atoms, optionally halogenated, or even perhalogenated;

said R₄ et R₅ moieties being able to form together with N, a 5 to 6 member carbon ring likely to additionally comprise at least one heteroatom selected amongst O, N and/or S in the ring, and/or likely to be substituted by 1 to 5 identical or different groups selected amongst —OH; —OR″; —O—COR″; —SH; —SR″; —S—COR″; —NH₂; —NHR″; —NH—COR″; —Hal (halogen); —CN; —COOR″; —COR″; with R″ representing a linear, branched or cyclic, saturated or unsaturated hydrocarbon moiety having 1 to 6 carbon atoms, optionally halogenated, or even perhalogenated.

In this definition, the mineral or organic acid salts of said compounds are also included, as well as the optical isomers thereof, in an isolated form or as a racemic mixture.

The expression linear, cyclic or branched hydrocarbon moiety means more particularly moieties of the alkyl, aryl, aralkyl, alkylaryl, alkenyl and alkynyl type.

The C₆H₅ group present in the R3 moiety is to be understood as an aromatic cyclic group.

Preferably, the Y moiety represents oxygen.

Preferably, the R1 moiety represents hydrogen or a linear or branched, saturated or unsaturated hydrocarbon moiety having 1 to 12, and more preferably, 1, 2, 3, 4, 5 or 6 carbon atoms, optionally substituted.

In particular, the substituants can be selected amongst —OH, —OR and/or —P(O)—(OR)₂ with R representing a linear, branched or cyclic, saturated or unsaturated hydrocarbon moiety having 1 to 6 carbon atoms, optionally halogenated, even perhalogenated.

Preferably, the R1 moiety represents a methyl, ethyl, propyl or isopropyl moiety, optionally substituted by a —OH or —P(O)—(OR)₂ group, with R representing methyl, ethyl, propyl, or isopropyl.

Preferably, the R2 moiety represents a linear, branched or cyclic, saturated or unsaturated hydrocarbon moiety having 1 to 12, preferably 1, 2, 3, 4, 5 or 6 carbon atoms, optionally substituted.

In particularly, the substituants can be selected amongst —OH and —OR with R representing a linear, branched or cyclic, saturated or unsaturated hydrocarbon moiety having 1 to 6 carbon atoms, optionally halogenated, even perhalogenated.

Preferably, the R2 moiety represents a methyl, ethyl, propyl, isopropyl, n-butyl, tert-butyl, or isobutyl moiety.

Preferably, the R3 moiety represents a moiety with the —C₆H_{(5-y′)4}—B_{y′} form, where y′=1, 2 or 3; or a moiety represented by the formula —A—C₆H_{(5-y)}—B_{y} where y=0, 1 or 2.

Preferably, A is a linear or branched, saturated or unsaturated divalent hydrocarbon moiety having 1 to 12 carbon atoms, optionally substituted.

The substituants for A are preferably selected amongst —CN; —COOR; —NO₂; —SO₂—OR; with R representing a linear, branched or cyclic, saturated or unsaturated hydrocarbon moiety having 1 to 6 carbon atoms, optionally halogenated, or even perhalogenated.

Preferably, B is a linear or branched, saturated or unsaturated hydrocarbon moiety having 1 to 12 carbon atoms, optionally substituted.

The substituants for B are preferably selected amongst —Hal (halogen, even perhalogen); —CN; —COOR; —NO₂; —SO₂—OR; with R representing a linear, branched or cyclic, saturated or unsaturated hydrocarbon moiety having 1 to 6 carbon atoms, optionally halogenated, or even perhalogenated.

Preferably, the R3 moiety represents a group selected amongst one of the following formulae:

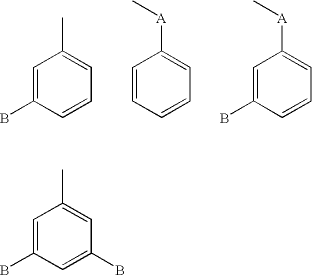

wherein A and B are defined as hereinabove.

More particulary, the A divalent moiety can be a methylene, an ethylene, a propylene.

The B moiety is preferably a methyl, ethyl, propyl or isopropyl moiety, substituted by one or more halogens, more particularly, chlorine, bromine, iodine or fluorine, and preferably completely halogenated (perhalogenated), such as perfluorated. One can more particularly mention the perfluoromethyl moiety (—CF3) as being most preferred.

Preferably, the X moiety represents a moiety selected amongst —OH or —OR₄ with R₄ representing a linear, cyclic or branched, saturated or unsaturated hydrocarbon moiety having 1 to 6 carbon atoms, optionally substituted.

The substituants can be selected amongst —OH and —OR with R representing a linear, branched or cyclic, saturated or unsaturated hydrocarbon moiety having 1 to 6 carbon atoms, optionally halogenated, even perhalogenated.

Preferably, the X moiety represents a moiety selected amongst —OH, —OCH₃, —OC₂H₅, —O—C₃H₇ or —OC₄H₉.

The most preferred compounds can include:

the {2-[acetyl-(3-trifluoromethyl-phenyl)-amino]-3-methyl-butyrylamino}acetic acid,

the ethyl {2-[acetyl-(3-trifluoromethyl-phenyl)-amino]-3-methylbutyrylamino}acetate,

the [2-(acetylbenzylamino)-3-methyl-butyrylamino]acetic acid,

the ethyl [2-(acetylbenzylamino)-3-methylbutyrylamino]acetate,

the ethyl (2-{benzyl-[(diethoxyphosphoryl)-acetyl]-amino}-3-methyl-butyrylamino]acetate.

The compounds according to the invention can be easily separated by the man of the art based on its general knowledge. A carboxylic acid, an aldehyde, an amino compound and an isonitrile can be reacted together according to Ugi's reaction.

Obviously, during the synthesis of the compounds according to the invention, and depending on the nature of the various moieties present on the starting compounds, the man of the art could take care to protect some substituants so that the latter do not interfere in the reaction course.

The compound amount to be used in the compositions according to the invention can be easily determined by the man of the art, depending on the nature of the compound being used, on the person to be treated and/or. the effect being sought. Generally, such an amount could range from 0.00001 to 20% in weight based on the total weight of the composition, more preferably from 0.001 to 10% in weight, and most preferably, from 0.5 to 1% in weight.

The association of compounds of the formula (I) with a compound reducing the hair loss and/or enhancing the hair regrowth can more particularly be used in a composition comprising a physiologically acceptable medium, more particularly in a cosmetic or pharmaceutical composition comprising consequently a cosmetically or pharmaceutically acceptable medium as well.

The physiologically acceptable medium wherein the compounds according to the invention could be used, as well as the constituents thereof, their amount, the galenic form of the composition and the preparation mode thereof, can be selected by the man of the art based on its general knowledge depending on the type of composition being sought.

Generally, such a medium can be anhydrous or aqueous, it can therefore comprise an aqueous phase and/or a fatty phase.

Preferred Hair Anti-loss Compounds According to the Invention

The compounds reducing the hair loss or enhancing the hair regrowth according to the invention are preferably agents improving the viability of the hair follicle.

The preferred compounds are vitamins, such as Biotin, Aminexil, minoxidil, KPV tripeptide and the salts thereof, sweet energetic agents, and ginseng.

Other preferred compounds comprise compounds systemically acting on the androgen metabolism, such as Finasteride®.

The hair anti-loss agents are preferably present in a concentration ranging from 0.01 to 10% in total weight of the composition, preferably from 0.1 to 5%.

The association of at least one N-acylaminoamide compound and at least one compound reducing the hair loss and/or enhancing the hair regrowth could be more particularly used, alone or in blend, in a composition comprising a physiologically acceptable medium, more particularly, in a cosmetic or pharmaceutical composition therefore comprising a cosmetically or pharmaceutically acceptable medium as well.

The physiologically acceptable medium wherein the compounds according to the invention could be used, as well as the constituents thereof, their amount, the galenic form of the composition and the preparation mode thereof, can be selected by the man of the art based on its general knowledge depending on the type of composition being sought.

Generally, such a medium can be anhydrous or aqueous, it can therefore comprise an aqueous phase and/or a fatty phase.

For an application on the skin, the composition could have the form, more particularly, of an aqueous or oily solution; of a dispersion of the lotion or serum type; of emulsions having a liquid or semi-liquid consistency of the milk type obtained through dispersion of a fatty phase in an aqueous phase (O/W) or inversely (W/O); of slurries or emulsions having a soft consistency of the cream or aqueous gel or anhydrous type; of microcapsules or microparticles; of vesicular dispersions of the ionic and/or non ionic type.

For an application on the hair, the composition could be in the form of aqueous, alcoholic or hydroalcoholic solutions; in the form of creams, gels, emulsions, foams; in the form of aerosol compositions also comprising a pressurized propellant.

The compositions according to the invention could have various forms commonly used is cosmetics or in dermatology for treating the hair scalp.

They can more particularly have the form of lotions, shampoos, foams, creams, gels, sticks, sprays, balms, powders, solid or liquid soaps.

The physiologically acceptable medium generally comprises water or a mixture of water and at least one physiologically acceptable organic solvent for a topic application purpose. Such solvents may include acetone, lower C₁-C₄ alcohols, such as ethanol, isopropyl alcohol, alkylene glycols, such as ethylene glycol, propylene glycol, ethylene glycol monomethyl, monoethyl or monobutyl ethers, propylene glycol and dipropylene glycol monoethylethers, short chain acid C₁-C₄ alkyl esters and polytetrahydrofuran ethers. When present, such solvents preferably account for 1 to 80% in weight of the total weight of the composition.

The medium could be thickened using thickening agents commonly used in cosmetics and pharmacy.

Those thickening agents may include more particularly cellulose and the derivates thereof such as cellulose ethers, heterobiopolysaccharides such as xanthane gum, scleroglucans, crosslinked or not crosslinked polyacrylic acids.

The thickening agents are preferably present in proportions ranging from 0.1 to 5% in weight approximately based on the total weight of the composition.

Depending on the various desired presentation forms of the compositions, the man of the art will be able to select the required compounds and builders commonly used for producing such compositions.

Amongst such builders, one can more particularly mention preservatives, stabilizers, pH regulators, osmotic pressure modifiers, emulsifiers, sun screens, antioxidants, perfumes, dyes, anionic, cationic, non ionic, amphoteric, zwitterionic surfactants, or the mixtures thereof, polymers, etc.

The compositions can further additionally contain, beside the particular association being the subject of the invention, compounds already known for slowing down the hair loss.

Preferably, the composition according to the invention could be used in anti-loss/regrowth care cosmetic compositions for alopecic areas specially when these are exposed to sunlight. Indeed, the relative decrease of the hair density occurring during alopecia contributes to a reduced UV protection—as a result, the inflammatory component of such an alopecia could be maximized through a large exposure to UV radiation.

Another object of the invention is also a method for cosmetically treating hair and/or hair scalp consisting in applying to it a composition such as defined hereinabove, with a view to reducing the loss thereof.

The preferred application mode consists in applying 0.1 to 20 g of the composition on all or a part of the hair scalp, with a frequency of one to two applications a day, for 1 to 7 days a week, and this for a 1 to 9 month time, to be renewed depending on the evolution of the alopecia progress.

When the composition has an aqueous form, more particularly the form of an aqueous dispersion, emulsion or solution, it could comprise an aqueous phase, which can contain water, flower water and/or mineral water.

Said aqueous phase could additionally comprise alcohols such as C₁-C₆ monoalcohols and/or polyols such as glycerol, butylene glycol, isoprene glycol, propylene glycol, polyethylene glycol.

When the composition according to the invention has the form of an emulsion, it could optionally additionally comprise a surfactant, preferably in an amount ranging from 0.01 to 30% in weight based on the total weight of the composition. The composition according to the invention could also comprise at least one co-emulsifier that could be selected amongst oxyethylenated sorbitan monostearate, fatty alcohols such as stearyl alcohol or cetyl alcohol, or fatty acid and polyol esters such as glyceryl stearate.

The composition according to the invention could also comprise a fatty phase, more particularly consisting in fatty species being liquid at 25° C., such as oils from animal, vegetable, mineral or synthetic origin, whether volatile or not; fatty species being solid at 25° C., such as waxes from animal, vegetable, mineral or synthetic origin; pasty fatty species; gums; the mixtures thereof.

The volatile oils are generally oils having at 25° C. a saturating vapour tension at least equal to 0.5 millibar (i.e. 50 Pa).

Amongst the components of the fatty phase, one can mention:

volatile cyclic silicones having from 3 to 8 silicon atoms, preferably from 4 to 6,

cyclocopolymers of the dimethylsiloxane/methylalkylsiloxane type;

volatile linear silicones having from 2 to 9 silicon atoms,

volatile hydrocarbon oils, such as isoparaffins and more particularly, isododecane and fluorinated oils,

poly(C₁-C₂₀)alkyl siloxanes and more particularly those having trimethylsilyl end groups, amongst which one can mention linear polydimethylsiloxanes and alkylmethylpolysiloxanes such as cethyldimethicone (referred to as CTFA),

silicones modified by aliphatic and/or aromatic groups, optionally fluorinated, or by functional groups such as hydroxyl, thiol and/or amine groups,

phenylated silicone oils,

oils from animal, vegetable or mineral origin, and more particularly animal or vegetable oils formed by fatty acid and polyol esters and in particular liquid triglycerides, for example, sun flower, corn, soya, marrow, grape seed, sesame, hazel—nut, apricot, almond or avocado oils; fish oils, glycerol tricaprocaprylate or vegetable or animal oils of the formula R₁COOR₂ wherein R₁ represents the residue of a higher fatty acid comprising from 7 to 19 carbon atoms and R₂ represents a branched hydrocarbon chain containing from 3 to 20 carbon atoms, for example, Purcellin oil; paraffin, petroleum jelly, perhydrosqualene, wheat germ, calophyllum, sesame, macadamia, grape seed, rapeseed, copra, peanut, palm, castor, jojoba, olive or cereal germ oils; fatty acid esters; alcohols; acetylglycerides; alcohol or polyalcohol octanoates, decanoates or ricinoleates; fatty acid triglycerides; glycerides,

fluorinated and perfluorinated oils,

silicone gums,

waxes from animal, vegetable, mineral or synthetic origin, such as microcrystalline waxes, paraffin, petrolatum, petroleum jelly, ozokerite, montan wax; beewax, lanolin and the derivates thereof; Candelilla, ouricury, camauba, Japan, cocoa butter waxes, cork or sugar cane fibre waxes; hydrogenated oils being concreted at 25° C., ozokerites, fatty esters and glycerides being concreted at 25° C.; polyethylene waxes and waxes obtained through Fischer-Tropsch synthesis; hydrogenated oils being concreted at 25° C.; lanolins, fatty esters being concreted at 25° C.; silicone waxes; fluorinated waxes.

As is known, the composition according to the invention could comprise the usual builders in the field being considered, such as hydrophilic or lipophilic gellants, hydrophilic or lipophilic additives, active ingredients, in particular, hydrophilic or lipophilic cosmetic or pharmaceutical, preservatives, antioxidants, solvents, perfumes, fillers, pigments, mother-of-pearls, UV filters, odour absorbers and dyes. Such builders, depending on their nature, could be introduced in the fatty phase, in the aqueous phase and/or in lipid spherules.

The nature and the amount of such builders could be selected by the man of the art, based on its general knowledge, so as to obtain the presentation form being sought for the composition. Anyway, the man of the art will take care to select all the optional complementary components and/or their amount such that the advantageous properties of the composition according to the invention should not be, or substantially not, altered by the contemplated addition.

The cosmetic or pharmaceutical compositions according to the invention could more particularly have the form of a composition designed for caring and/or treating ulcerated areas or having been subjected to a cutaneous stress or microstress, more particularly generated by an exposure to UV and/or a contact with an irritating product.

Now, the compositions according to the invention could more particularly have the following form:

a care, treatment, cleaning or protective product for the hair scalp, such as a hair composition, and in particular, a sun protective cream or gel; a hair scalp care composition, more particularly for hair anti-loss or regrowth; an antiparasitic or soothing shampoo. Another object of the invention is a method for cosmetically treating hair and/or hair scalp consisting in applying a composition such as defined hereinabove, with a view to reducing hair loss.

The method for cosmetic treatment of the invention could be implemented more particularly applying cosmetic compositions such as defined herein above, by means of the common using technique for such compositions. For example : applying creams, gels, serums, lotions, shampoos, cleansing milks or sun or after sun protective compositions on the skin or on dry hair; applying a lotion for the hair scalp on wet hair.

The preferred application mode consists in applying 1 to 20 g of the composition on all or part of the hair scalp, with a frequency of one to two applications a day, for 1 to 7 days a week, and this for a 1 to 9 month time, to be renewed depending on the extent of the exposure to the stress agent and depending on the alopecia degree.

The following examples are meant for illustrating the invention without in any way limiting its scope.

### EXAMPLE 1

Preparation of ethyl {2-[acetyl-(3-trifluoromethylphenyl)-amino]-3-methylbutyrylamino}acetate of the formula:

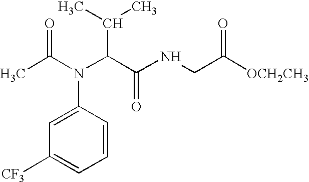

0.63 ml of isobutyraldehyde and 1 ml of trifluoromethylamine (1.15 eq) are mixed in 15 ml of methanol, under stirring. This is allowed to react for 15 minutes at 20° C., then 0.46 ml of acetic acid (1. 15 eq) are added and this is allowed to react 10 minutes at 20° C. 0.8 ml of ethyl isocyanoacetate (1 eq) are then added at 95% (1 eq) and this allowed to react 48 hours at 20° C.

The reaction medium is concentrated with a rotovapor and the residue is purified on a silica column (eluant: heptane: 3/ethyl acetate: 7; Rf=0.5).

2.45 g of compound are obtained in the form of a waxy solid, whence a 91% yield.

¹H NMR (200 MHz, CDCl3) δ ppm: 0.9 (6H;q); 1.3 (3H;t); 1.8 (3H;s), 2.3 (1H;m), 4.0 (2H,q), 4.2 (2H,Q), 4.4 (2H;d), 7.3 (1H;t), 7.5 (4H;m).

### EXAMPLE 2

Preparation of {2-[acetyl-(3-trifluoromethyl-phenyl)-amino]-3-methyl-butyrylamino}acetic acid of the formula:

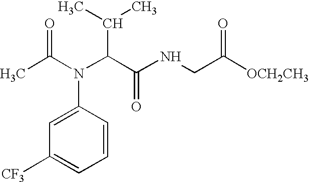

2 g of compound as prepared in example 1 are solubilized in 30 ml of acetone. 30 ml of 2N soda are added and this is allowed to react for 6 hours at 20° C. The reaction medium is concentrated with a rotovapor. The residual aqueous phase is acidified at pH 2 through the addition of concentrated HCl, followed by extraction with CH2Cl₂.

The organic phase is dry concentrated after drying on sodium sulphate.

A residue is obtained which is solubilized by a mixture of basic water at 10% ethanol, then again acidified by concentrated HCl at pH 2. A new extraction follows with CH₂Cl₂ and the organic phase is dried on sodium sulphate, filtered and dry concentrated under vacuum with a rotovapor.

1.3 g of compound are obtained in the form of a slightly light brown solid, whence a 70% yield.

¹H NMR (200 MHz, DMSO) δ ppm: 0.9 (6H;q); 3.7 (2H;m); 1.8 (4H;m), 4.8 (2H;d), 7.6 (4H,m), 8.4 (1H;t), 12.5 (1H;s).

### EXAMPLE 3

The anti-elastase activity of compounds according to the invention has been determined relative to human leukocyte elastase (HLE).

The test is performed as follows.

A Me-OSAAPV-p-NA (methyl-O-succinate alanine alanine proline valine-p-nitroanilide) substrate is allowed to incubate, at 37° C., for 60 minutes, on which it is applied HLE (40 milli-units per ml) and 0.1% of the compound to be tested.

The inhibition % of the control elastase activity is then determined through spectrophotometry.

The compounds to be tested are as follows:

compound A: {2-[acetyl-(3-trifluoromethyl-phenyl)-amino]-3-methyl-butyrl amino}acetic acid,

compound B: ethyl (2-{benzyl-[(diéethoxyphosphoryl)-acetyl]-amino}-3-méthylbutyrylamino) acetate,

compound C: [2-(acetylbenzylamino)-3-methyl-butyrylamino]acetic acid,

compound D: ethyl [2-(acetylbenzylamino)-3-methyl-butyryl-amino]acetate.

The following results are obtained:

| | Inhibition % of the control elastase |
|---|---|
| Compound (concentration: 0.1%) | activity |
| Compound A | 67% |
| Compound B | 17% |
| Compound C | 20% |
| Compound D | 13% |

The same way, the inhibition % of the control elastase activity is determined for the compound A at different concentrations:

The following results are obtained:

| Concentration of | Inhibition % of the |
|---|---|
| compound A | control elastase activity |
| 0.01% | 53% |
| 0.05% | 50% |
| 0.1% | 68% |
| 0.2% | 68% |

Compound A therefore leads to a strong inhibition of the elastase activity, even in a low amount.

### EXAMPLE 4

The ex vivo activity of the compound in example 2 has been evaluated on surviving human skins treated by human leukocyte elastase (HLE).

The test is performed as follows.

Fresh cuts from human skins, originating from 2 different donors, are treated for 2 hours, at 20° C., by 20 μl of a buffer solution (pH 7.4) optionally comprising 10 μg/ml of HLE and optionally 0.1% of the compound to be tested, optionally previously put in a solution in ethanol.

The elastic fibres are coloured in blue using (+) catechin and are morphometrically quantified by computer assisted picture analysis. The surface percentage of average dermis occupied by the elastic fibres was evaluated this way.

The following results are obtained:

Surface % occupied
by the elastic fibres

| | Skin 1 | Skin 2 |
|---|---|---|
| Control (non treated skin) | 12.7% | 15.25% |
| HLE treated skin | 4.85% | 6.85% |
| Skin treated with HLE + compound | 13.95% | 11.85% |
| in example 2 | | |

It can therefore be seen that the compound according to the invention generates a significant protection of the skins towards the elastic fibre destruction induced by elastase.

### EXAMPLE 5

The ex vivo activity of the compound in example 2 has been evaluated on surviving human skins treated by human leukocyte elastase (HLE).

The test is performed as follows.

Fragments of normal human skin, originating from 3 different donors, are placed in inserts positioned in culture wells. Some antibiotic supplemented culture medium is added in the bottom of the wells. A passage occurs through slow diffusion between both compartments by means of a porous membrane (pore size: 12 μm).

The culture medium is renewed every three days.

Optionally 0.5 μg of HLE per ml of culture medium are optionally added on the skin fragments.

Also are added, every other day, 5 μl of the compound to be tested, previously put in a solution at 0.2% in weight in ethanol.

The skins are kept surviving for 10 days at 37° C.

The elastic fibres are coloured in blue using (+) catechin and are morphometrically quantified by computer assisted picture analysis. The surface percentage of average dermis occupied by the elastic fibres was evaluated this way.

The following results are obtained:

| | Surface % occupied by the elastic |
|---|---|
| | fibres |
| Control (non treated skin) | 7.4% |
| HLE treated skin | 5.1% |
| Skin treated with HLE + compound | 7.1% |
| in example 2 | |

It can therefore be seen that the compound according to the invention generates a significant protection of the skins towards the elastic fibre destruction induced by elastase.

### EXAMPLE 6

The activity of the compound in example 2 has been evaluated on surviving UVA irradiated human skins (8 J/cm²).

The test is performed as follows.

Fragments of normal human skin, originating from four different donors, are placed in inserts positioned in culture wells. Some antibiotic supplemented culture medium is added in the bottom of the wells. A passage occurs through slow diffusion between both compartments by means of a porous membrane (pore size: 12 μm).

The culture medium is renewed every three days.

On the skin fragments, every two days, are added 5 μl of the compound to be tested, previously put in a solution at 0.2% in ethanol.

The skins are kept surviving for 7 days at 37° C.

The skins are irradiated once at 8 J/cm² at 37° C. (Vilbert-Lourmat lamp, RMX-3W).

The elastic fibres are coloured in blue using (+) catechin and are morphometrically quantified by computer assisted picture analysis. The surface percentage of average dermis occupied by the elastic fibres was evaluated this way.

The following results are obtained:

| | Morphometric | Morphometric |
|---|---|---|
| | analysis of the elastic | analysis of the |
| | fibres (superficial | collagen (superficial |
| | dermis) | dermis) |
| Non treated skin | 6.75% | 87% |
| UVA treated skin (8 J/cm²) | 3.9% | 81% |
| Skin treated with UVA | 6.8% | 92% |
| (8 J/cm²) + compound | | |

It can be seen that the compound according to the invention does have an activity towards the elastic fibre damage in the superficial dermis of UV irradiated skins.

### EXAMPLE 7

Hair Lotion

The following lotion is prepared in a conventional way (% in weight):

| | |
|---|---|
| Compound from example 1: | 1% |
| Propylene glycol | 23% |
| Ethanol | 55% |
| Hydrocortisone | 0.1% |
| Minoxidil | 1.5% |

Such a lotion can be applied on the scalp of alopecic individuals, in order to prevent the UV effects, before and/or after exposure to sunlight.

### EXAMPLE 8

Anti-loss Lotion

The following lotion is prepared in a conventional way (% in weight):

| | |
|---|---|
| Compound from example 2: | 1% |
| Propylene glycol | 23% |
| Ethanol | 55% |
| Aminexil | 1.5% |
| Water | qsp 100% |

Such an anti-hair loss lotion can be applied on the scalp of alopecic individuals.

1. A cosmetic or dermatological composition characterized in that it comprises an association between an elastase inhibiting compound of the N-acylaminoamide family and at least one compound reducing the hair loss and/or enhancing the hair regrowth.

2. A composition according to claim 1, characterized in that the compound, the inhibiting compound of the N-acylaminoamide family is a compound represented by the following formula (I): 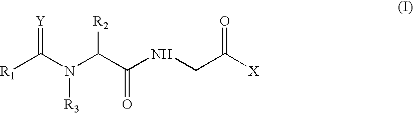
wherein
the Y moiety represents O or S,
the R1 moiety represents:
(i) a hydrogen atom;
(ii) an unsaturated or saturated, cyclic, branched or linear, hydrocarbon moiety, having 1 to 18 carbon atoms, optionally substituted by 1 to 5 identical or different groups selected amongst —OH; —OR; —O—COR; —SH; —SR; —S—COR; —NH₂; —NHR; —NRR′; —NH—COR; —Hal (halogen); —CN; —COOR; —COR; —P(O)—(OR)₂; —SO₂—OR;
with R and R′ representing, independently from one another, a linear, branched or cyclic, saturated or unsaturated hydrocarbon moiety having 1 to 6 carbon atoms, optionally halogenated, or even perhalogenated, said R and R′ moieties being able to form together with N, a 5 to 6 member carbon ring likely to additionally comprise one heteroatom selected amongst O, N and/or S in the ring, and/or likely to be substituted by 1 to 5 groups, whether identical or different, selected amongst —OH; —OR″; —O—COR″; —SH; —SR″; —S—COR″; —NH₂; —NHR″; —NH—COR″; —Hal (halogen); —CN; —COOR″; —COR″; with R″ representing, independently from one another, a linear, branched or cyclic, saturated or unsaturated hydrocarbon moiety having 1 to 6 carbon atoms, optionally halogenated, or even perhalogenated;
(iii) a moiety selected amongst the following moieties: —OR;. —NH₂; —NHR; —NRR′; —NH—COR; —COOR; —COR; with R and R′ representing, independently from one another, a linear, branched or cyclic, saturated or unsaturated hydrocarbon moiety having 1 to 6 carbon atoms, optionally halogenated, or even perhalogenated; said R and R′ moieties being able to form together with N, a 5 to 6 member carbon ring likely to additionally comprise at least one heteroatom selected amongst O, N and/or S in the ring, and/or likely to be substituted by 1 to 5 identical or different groups selected amongst —OH; —OR″; —O—COR″; —SH; —SR″; —S—COR″; —NH₂; —NHR″; —NH—COR″; —Hal (halogen); —CN; —COOR″; —COR″; with R″ representing a linear, branched or cyclic, saturated or unsaturated hydrocarbon ring having 1 to 6 carbon atoms, optionally halogenated, or even perhalogenated;
the R2 moiety represents a linear, branched or cyclic, saturated or unsaturated hydrocarbon moiety having 1 to 18 carbon atoms; optionally substituted by 1 to 5 identical or different groups selected amongst —OH; —OR; —O—COR; —SH; —SR; —S—COR; —NH₂; —NHR; —NRR′; —NH—COR; —Hal (halogen); —CN; —COOR; —COR;
with R and R′ representing, independently from each other, a linear, branched or cyclic, saturated or unsaturated hydrocarbon moiety having 1 to 6 carbon atoms, optionally halogenated, or even perhalogenated;
said R et R′ moieties being able to form together with N, a 5 to 6 member carbon ring likely to additionally comprise at least one heteroatom selected amongst O, N and/or S in the ring, and/or likely to be substituted by 1 to 5 identical or different groups selected amongst —OH; —OR″; —O—COR″; —SH; —SR″; —S—COR″; —NH₂; —NHR″; —NH—COR″; —Hal (halogen); —CN; —COOR″; —COR″; with R″ representing a linear, branched or cyclic, saturated or unsaturated hydrocarbon moiety having 1 to 6 carbon atoms, optionally halogenated, or even perhalogenated;
the R3 moiety represents a moiety selected amongst of the formula (II) or (III):
—A—C₆H_{(5-y)—B}_{y} (II)
—C₆H_{(5-y′)—B}_{y′} (III)
wherein
y is an integer between 0 and 5 inclusive, and y′ is an integer between 1 and 5 inclusive;
A is a linear or branched, saturated or unsaturated divalent hydrocarbon moiety having 1 to 18 carbon atoms;
optionally substituted by 1 to 5 identical or different groups selected amongst —OH; —OR; —O—COR; —SH; —SR; —S—COR; —NH₂; —NHR; —NRR′; —NH—COR; —Hal (halogen or even perhalogen); —CN; —COOR; —COR; —NO₂; —S0₂—OR;
with R and R′ representing, independently from each other, a linear, branched or cyclic, saturated or unsaturated hydrocarbon moiety having 1 to 6 carbon atoms, optionally halogenated, or even perhalogenated;
said R et R′ moieties being able to form together with N, a 5 to 6 member carbon ring likely to additionally comprise at least one heteroatom selected amongst O, N and/or S in the ring, and/or likely to be substituted by 1 to 5 identical or different groups selected amongst —OH; —OR″; —O—COR″; —SH; —SR″; —S—COR″; —NH₂; —NHR″; —NH—COR″; —Hal (halogen); —CN; —COOR″; —COR″; with R″ representing a linear, branched or cyclic, saturated or unsaturated hydrocarbon moiety having 1 to 6 carbon atoms, optionally halogenated, or even perhalogenated;
B is a linear or branched, saturated or unsaturated hydrocarbon moiety having 1 to 18 carbon atoms;
optionally substituted by 1 to 5 identical or different groups selected amongst —OH; —OR; —O—COR; —SH; —SR; —S—COR; —NH₂; —NHR; —NRR′; —NH—COR; —Hal (halogen or perhalogen); —CN; —COOR; —COR; —NO₂; —SO₂—OR; with R and R′ representing, independently from each other, a linear, branched or cyclic, saturated or unsaturated hydrocarbon moiety having 1 to 6 carbon atoms, optionally halogenated, or even perhalogenated;
said R et R′ moieties being able to form together with N, a 5 to 6 member carbon ring likely to additionally comprise at least one heteroatom selected amongst O, N and/or S in the ring, and/or likely to be substituted by 1 to 5 identical or different groups selected amongst —OH; —OR″; —O—COR″; —SH; —SR″; —S—COR″; —NH₂; —NHR″; —NH—COR″; —Hal (halogen); —CN; —COOR″; —COR″; with R″ representing a linear, branched or cyclic, saturated or unsaturated hydrocarbon moiety having 1 to 6 carbon atoms, optionally halogenated, or even perhalogenated;
the X moiety represents a moiety selected amongst —OH; —OR₄; —NH₂; —NHR₄; —NR₄R₅, —SR₄; —COOR₄; —COR₄;
with R₄ and R₅ representing, independently from one another, a linear, cyclic or branched, saturated or unsaturated hydrocarbon moiety having 1 to 6 carbon atoms, optionally substituted by 1 to 5 identical or different groups selected amongst —OH; —OR; —O—COR; —SH; —SR; —S—COR; —NH₂; —NHR; —NRR′; —NH—COR; —Hal (halogen or even perhalogen); —CN; —COOR; —COR; with R and R′ representing, independently from each other, a linear, branched or cyclic, saturated or unsaturated hydrocarbon moiety having 1 to 6 carbon atoms, optionally halogenated, or even perhalogenated; said R et R′ moieties being able to form together with N, a 5 to 6 member carbon ring likely to additionally comprise at least one heteroatom selected amongst O, N and/or S in the ring, and/or likely to be substituted by 1 to 5 identical or different groups selected amongst —OH; —OR″; —O—COR″; —SH; —SR″; —S—COR″; —NH₂; —NHR″; —NH—COR″; —Hal (halogen); —CN; —COOR″; —COR″; with R″ representing a linear, branched or cyclic, saturated or unsaturated hydrocarbon moiety having 1 to 6 carbon atoms, optionally halogenated, or even perhalogenated;
said R₄ et R₅ moieties being able to form together with N, a 5 to 6 member carbon ring likely to additionally comprise at least one heteroatom selected amongst O, N and/or S in the ring, and/or likely to be substituted by 1 to 5 identical or different groups selected amongst —OH; —OR″; —O—COR″; —SH; —SR″; —S—COR″; —NH₂; —NHR″; —NH—COR″; —Hal (halogen); —CN; —COOR″; —COR″; with R″ representing a linear, branched or cyclic, saturated or unsaturated hydrocarbon moiety having 1 to 6 carbon atoms, optionally halogenated, or even perhalogenated.
the mineral or organic acid salts thereof, its optical isomers, in an isolated form or as a racemic mixture.

3. A composition according to claim 2, wherein the compound of formule (I) is such that:
the Y moiety represents oxygen, and/or
the R1 moiety represents hydrogen or a linear or branched, saturated or unsaturated hydrocarbon moiety, having from 1 to 12, and more preferably, 1, 2, 3, 4, 5 or 6 carbon atoms, optionally substituted and/or
the R1 substituants can be selected amongst —OH, —OR and/or —P(O)—(OR)₂ with R representing a linear, branched or cyclic, saturated or unsaturated hydrocarbon moiety having from 1 to 6 carbon atoms, optionally halogenated, even perhalogenated and/or
the R2 moiety represents a linear, branched or cyclic, saturated or unsaturated hydrocarbon moiety having from 1 to 12, preferably 1, 2, 3, 4, 5 or 6 carbon atoms, optionally substituted and/or
the substituants for R2 are selected amongst —OH and —OR with R representing a linear, branched or cyclic, saturated or unsaturated hydrocarbon moiety having 1 to 6 carbon atoms, optionally halogenated, even perhalogenated; and/or
the R3 moiety represents a moiety represented by the formula —C₆H_{(5-y)}—B_{y} wherein y′=1, 2 or 3; or a moiety represented by the formula —A—C₆H_{(5-y)}—B_{y} wherein y=0, 1 or 2 and/or
the A moiety for R3 is a linear or branched, saturated or unsaturated divalent hydrocarbon moiety having 1 to 12 carbon atoms, optionally substituted and/or
the B moiety for R3 is a linear or branched, saturated or unsaturated hydrocarbon moiety having 1 to 12 carbon atoms, optionally substituted; and/or
the substituants for A and/or B are selected amongst —Hal (halogen, even perhalogen); —CN; —COOR; —NO₂; —SO₂—OR; with R representing a linear, branched or cyclic, saturated or unsaturated hydrocarbon moiety having 1 to 6 carbon atoms, optionally halogenated, or even perhalogenated and/or
the X moiety represents a moiety selected amongst —OH or OR₄ with R4 representing a linear, cyclic or branched, saturated or unsaturated hydrocarbon moiety having 1 to 6 carbon atoms, optionally substituted and/or
the R4 substituants for X are selected amongst —OH and —OR with R representing a linear, branched or cyclic, saturated or unsaturated hydrocarbon moiety having 1 to 6 carbon atoms, optionally halogenated, even perhalogenated.

4. A composition according to any of claims 2 or 3, wherein the compound representing by formula (I) is such that:
the R1 moiety represents a methyl, ethyl, propyl or isopropyl moiety optionally substituted by a —OH or —P(O)—(OR)₂ group with R representing methyl, ethyl, propyl or isopropyl; and/or
the R2 moiety represents a methyl, ethyl, propyl, isopropyl, n-butyl, butyl, tert-butyl or isobutyl; and/or
the R3 moiety represents a group selected amongst one of the following formulae: 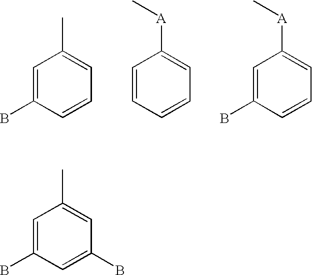
wherein the A divalent moiety is a methylene, an ethylene, a propylene or a propylene and/or the B moiety is a methyl, ethyl, propyl or isopropyl moiety, substituted by one or more halogens, more particularly, chlorine, bromine, iodine or fluorine, and preferably completely halogenated (perhalogenated), such as perfluorated, more particularly the perfluoromethyl moiety (—CF3),
the X moiety represents a moiety selected amongst —OH or—OR₄ with R4 representing a linear, cyclic or branched, saturated or unsaturated hydrocarbon moiety having 1 to 6 carbon atoms, optionally substituted.
The substituants can be selected amongst —OH, —OCH₃, —OC₂H₅, —O—C₃H₇ or —OC₄ H₉.

5. A composition according to any of claims 2 to 4, wherein the compound represented by formula (I) is selected amongst the following compounds:
the {2-[acetyl-(3-trifluoromethyl-phenyl)-amino]-3methyl-butyrylamino}acetic acid,
the ethyl {2-[acetyl-(3-trifluoromethyl-phenyl)-amino]-3-methyl-butyrylamino}acetate,
the [2-(acetyl-benzyl-amino)-3-methyl-butyrylamino] acetic acid,
the ethyl [2-(acetyl-benzyl-amino)-3-methyl-butyrylamino] acetate,
the ethyl (2-{benzyl-[(diethoxy-phosphoryl)-acetyl]-amino}-3-methyl-butyrylamino] acetate.

6. A composition according to any of preceding claims, wherein the elastase inhibiting compound from the N-acylaminoamide family is present in an amount ranging from 0.00001 to 20 % in weight based on the total weight of the composition, more preferably from 0.001 to 10% in weight, and most preferably, from 0.5 to 1% in weight.

7. A composition according to any of preceding claims, characterized in that the compound for reducing the hair loss or enhancing the hair regrowth is selected amongst agents improving the viability of the hair follicle.

8. A composition according to any of claims 1 to 6, characterized in that the compound for limiting the hair loss or enhancing the hair regrowth is selected amongst vitamins, sweet energetic agents and ginseng.

9. A composition according to any of claims 1 to 6, characterized in that the compound for reducing the hair loss or enhancing the hair regrowth is selected amongst Biotin, Aminexil, minoxidil, KPV tripeptide and the salts thereof.

10. A composition according to one of claims 1 to 6, characterized in that the compound for reducing the hair loss or enhancing the hair regrowth is selected amongst compounds systemically acting on the androgen metabolism.

11. A composition according to any of claims 1 to 6, characterized in that the compound for reducing the hair loss or enhancing the hair regrowth is preferably present in a concentration ranging from 0.01 to 10% in total weight of the composition, preferably from 0.1 to 5%.

12. A method for cosmetically treating the hair and/or the hair scalp, characterized in that a composition according to any of claims 1 to 11 is applied on the hair and/or on the hair scalp.
